(19) United States
(12) Patent Application Publication
(10) Pub. No.: US 2022/0387999 A1
(43) Pub. Date: Dec. 8, 2022

(54) ACOUSTIC-DIELECTROPHORETIC TRANSDUCER (ADEPT) FOR HIGH THROUGHPUT AND PRECISION PARTICLE SORTING

(71) Applicant: THE REGENTS OF THE UNIVERSITY OF CALIFORNIA, Oakland, CA (US)

(72) Inventors: Abraham P. Lee, Irvine, CA (US); Gisela Lin, Irvine, CA (US); Ruoyu Jiang, Irvine, CA (US); Mohammad Aghaamoo, Irvine, CA (US); Yu-Hsi Chen, Irvine, CA (US); Braulio Cárdenas Benítez, Irvine, CA (US)

(21) Appl. No.: 17/891,889

(22) Filed: Aug. 19, 2022

Related U.S. Application Data
(63) Continuation-in-part of application No. 17/340,581, filed on Jun. 7, 2021.
Application No. 17/340,581 is a continuation-in-part of application No. 16/547,152, filed on Aug. 21, 2019, now Pat. No. 11,052,395.
Application No. 16/547,152 is a continuation-in-part of application No. PCT/US21/27945, filed on Apr. 19, 2021.
(60) Provisional application No. 63/234,931, filed on Aug. 19, 2021.
(60) Provisional application No. 62/720,829, filed on Aug. 21, 2018.
(60) Provisional application No. 63/011,426, filed on Apr. 17, 2020.

(51) Int. Cl. B01L 3/00; C12Q 1/6806

(52) U.S. Cl. CPC B01L 3/502715; B01L 3/502761; B01L 3/50273; C12Q 1/6806; B01L 2400/0415; B01L 2400/0478; B01L 2400/0436

(57) ABSTRACT

The present invention is directed to systems and devices that allow for separation of cells based on size and electric properties and for high-throughput cell sorting. The system may comprise a microfluidic platform having a main microfluidic channel and cavity acoustic transducers (CATs). The microfluidic platform may be coupled to an external acoustic source. The system may further comprise a fluid disposed through the main microfluidic channel comprising cells having different sizes and electric properties. The fluid may intersect the CATs to form one or more interfaces. The system may further comprise electrodes underneath the microfluidic platform. The CATs may oscillate the interfaces to produce one or more microstreaming vortices, such that each microstreaming vortex is capable of selectively trapping cells based on size. The set of electrodes may apply an AC to cause the cells to move relative to the set of electrodes based on electric properties.

### CROSS-REFERENCES TO RELATED APPLICATIONS

This application is a non-provisional and claims benefit of U.S. Provisional Application No. 63/234,931 filed Aug. 19, 2021, the specification of which is incorporated herein in its entirety by reference.

This application is a continuation-in-part and claims benefit of U.S. application Ser. No. 17/340,581 filed Jun. 7, 2021, which is a continuation-in-part and claims benefit of U.S. Non-Provisional application Ser. No. 16/547,152 filed Aug. 21, 2019, now U.S. Pat. No. 11,052,395, which claims benefit of U.S. Provisional Application No. 62/720,829 filed Aug. 21, 2018, the specifications of which are incorporated herein in their entirety by reference.

This application is a continuation-in-part and claims benefit of PCT Application No. PCT/US2021/027945 filed Apr. 19, 2021, which claims benefit of U.S. Provisional Application No. 63/011,426 filed Apr. 17, 2020, the specification of which are incorporated herein in their entirety by reference.

### FIELD OF THE INVENTION

The present invention is directed to devices that allow for the separation of key cellular blood components of the immune system, directly applicable to the development of medicines for the treatment of infectious disease and cancer, as well as the separation of cells of interest in suspension from a heterogeneous population.

### BACKGROUND OF THE INVENTION

The isolation of key cell types in suspension from heterogeneous mixtures is an essential preparative step in medicine and biotechnology. For example, applications such as liquid biopsy, cancer immunotherapy, and cell manufacturing processes require high throughput and fine separation of cell subpopulations of the immune system. While the traditionally used density-based gradient separation methods provide enough cell numbers for many applications, the cell pelleting process often results in undesired ex-vivo immune cell activation at suboptimal isolation sensitivity. Other bulk cell centrifugation methods, such as counterflow centrifugation elutriation, have clinically relevant isolation yields, but rely solely on physical properties such as cell density and therefore lack specificity towards cell surface composition.

Magnetic activated cell sorting methods, on the other hand, solve the specificity issue by tagging a subpopulation of cells with antibodies conjugated to magnetic beads, which are then collected to achieve the separation. However, this technique requires the interaction of beads and cells and requires expensive reagents to operate. Alternative high throughput, label-free solutions to this problem include inertial microfluidics, and acoustofluidic techniques, yet these methods are prone to size-biased discrimination, preventing, for instance, the sensitive separation of equally sized cell subpopulations. Thus, there is a need for the development of label-free, high throughput sorting techniques that simultaneously maximize cell viability and purity, while minimizing size bias and non-specific interactions.

### BRIEF SUMMARY OF THE INVENTION

It is an objective of the present invention to provide devices that allow for the separation of key cellular blood components of the immune system, directly applicable to the development of medicines for the treatment of infectious disease and cancer, as well as the separation of cells of interest in suspension from a heterogeneous population, as specified in the independent claims. Embodiments of the invention are given in the dependent claims. Embodiments of the present invention can be freely combined with each other if they are not mutually exclusive.

The present invention features a microfluidic device that combines two key technologies to achieve high throughput sorting of cells in suspension. First, by creating flow vortices within the microfluidic chip, select sizes of cells may be trapped. Afterward, an array of electrodes may be used to exert a force to discriminate between cells of equal size, yet with different electrical properties. As an example, this device may be used to purify cell mixtures containing different subpopulations with overlapping sizes. This would be useful for applications where one wishes to extract specific cell types for bioengineering while maximizing cell viability in the process.

The present invention features a system for high-throughput cell sorting. The system may comprise a microfluidic platform that may comprise a main microfluidic channel, and one or more cavity acoustic transducers (CATs). The one or more CATs may be dead-end channels coupled to the main microfluidic channel. The microfluidic platform may be coupled to an external acoustic source. The system may further comprise a fluid disposed through the main microfluidic channel comprising cells having different sizes and different electric properties. The fluid may intersect the CATs to form one or more interfaces. The system may further comprise a set of electrodes disposed underneath the microfluidic platform. The set of electrodes may be configured to apply an alternating current (AC) to the cells. The CATs may be configured to oscillate the interfaces to produce one or more microstreaming vortices, such that each microstreaming vortex is capable of selectively trapping cells based on size. Applying the AC to the cells may cause the cells to move relative to the set of electrodes based on electric properties through dielectric polarization.

The present invention features a high-throughput method for cell sorting. The method may comprise providing a microfluidic platform. The microfluidic platform may comprise a main microfluidic channel and CATs. The one or more CATs may be dead-end channels coupled to the main microfluidic channel. The microfluidic platform may be coupled to an external acoustic source. The method may further comprise providing a set of electrodes disposed underneath the microfluidic platform. The method may further comprise flowing a fluid through the main microfluidic channel comprising cells having different sizes and different electric properties. The fluid may intersect the CATs to form one or more interfaces. The method may further comprise applying acoustic energy to the CATs via the external acoustic source to oscillate the interfaces. Oscillating the interfaces may produce one or more microstreaming vortices such that each microstreaming vortex is capable of selectively trapping cells based on size. The method may further comprise applying, by the set of electrodes, an AC to the cells. Applying the AC may cause the cells to move relative to the set of electrodes based on electric properties through dielectric polarization.

One of the unique and inventive technical features of the present invention is the application of both LCAT-induced microvortices and dielectrophoresis to a plurality of cells in a microfluidic platform. Without wishing to limit the invention to any theory or mechanism, it is believed that the technical feature of the present invention advantageously provides for the high-throughput sorting of cells based on both size and electric properties. None of the presently known prior references or work has the unique inventive technical feature of the present invention.

Furthermore, the inventive technical feature of the present invention is counterintuitive. The reason that it is counterintuitive is because it contributed to a surprising result. One skilled in the art would expect that the high hydrodynamic forces on the cells from the microvortices would be too strong to counterbalance with DEP forces to control the position and sort them out. Surprisingly, the present invention uses DEP forces not to completely halt particles but rather to change their location within the microfluidic chip so that they experience different hydrodynamic forces. The process results in the entrapment of larger cells while allowing the flow of cells of finite smaller sizes, which are then sorted by electric properties only. Thus, the inventive technical feature of the present invention contributed to a surprising result and is counterintuitive.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a microfluidic channel with CATs and bottom electrodes for DEP manipulation of particles.

FIG. 1B shows LCAT-DEP operation with vortices induced by the piezo transducer resulting in size-based particle trapping.

FIG. 1C shows a perspective view of the LCAT-DEP device, illustrating the bottom electrodes.

FIG. 1D shows a side view of the bottom electrodes, displaying the electric field lines and their effect on a particular subtype of Cell B. As illustrated, cells are attracted to the electrodes through positive DEP which is a result of their intrinsic dielectric properties, thus allowing the discrimination between equally sized cells. In some embodiments, the cells may comprise one or more cell subtypes.

FIG. 2 shows a flowchart of a high-throughput method for cell sorting.

FIG. 3A shows a microfluidic device placed atop a piezoelectric transducer, which is used to drive the oscillation of the air/liquid interfaces and the formation of vortices that trap incoming particles of a select size range.

FIG. 3B shows a photo of the microfluidic device, showing the microchannel with electrodes at the bottom.

FIG. 3C shows an example of cells being trapped in the microfluidic vortices.

### DETAILED DESCRIPTION OF THE INVENTION

Following is a list of elements corresponding to a particular element referred to herein:
100 system
110 microfluidic platform
120 main channel
130 cavity acoustic transducer (LCAT)
140 external acoustic source
150 fluid
160 plurality of cells
180 interfaces
190 microstreaming vortices
200 electrodes

As used herein, the term “subset of the plurality of cells” refers to any selection of cells from the plurality of cells with a number of cells less than or equal to the number of cells in the plurality of cells. Thus, the subset may comprise every cell in the plurality of cells, zero cells, or any number of cells in between.

As used herein, Cavity Acoustic Transducers (CATs) are simple on-chip actuators that are easily fabricated and can be actuated using a battery-operated portable electronics platform. CATs are dead-end channels that are in the same plane laterally with respect to the microchannels. In some embodiments, the CATs require no additional fabrication steps other than those needed to produce a single layer or multilayer device. When the device is filled with liquid, CATs trap bubbles creating an interface that can be excited using an external acoustic source such as a piezoelectric transducer. The interface generated by an LCAT may be selected from a group comprising a gas-liquid interface, a liquid-liquid interface, a lipid membrane, a polymer membrane, a nano-particle membrane, or a combination thereof. In some embodiments, the liquid-liquid interface may comprise a plurality of immiscible liquids. As used herein, the term “immiscible liquids” refers to a set of liquids that are incapable of mixing (e.g. water and a hydrophobic liquid such as oil). In other embodiments, the liquid-liquid interface may comprise a thin physical barrier between the liquids, in which case the liquids may be immiscible or miscible. As used herein, the term “thin” refers to a membrane with a width of 2 to 100 nm. In some embodiments, the lipid membrane may comprise a lipid bilayer. In some embodiments, the polymer membrane may comprise a synthetically created membrane capable of enacting a driving force (e.g. pressure or concentration gradients) on particles on either side of the polymer membrane.

As used herein, “air” may refer to a gas or mixture of gasses, such as atmospheric air, oxygen, nitrogen, helium, neon, argon, an inert gas, or a reactive gas.

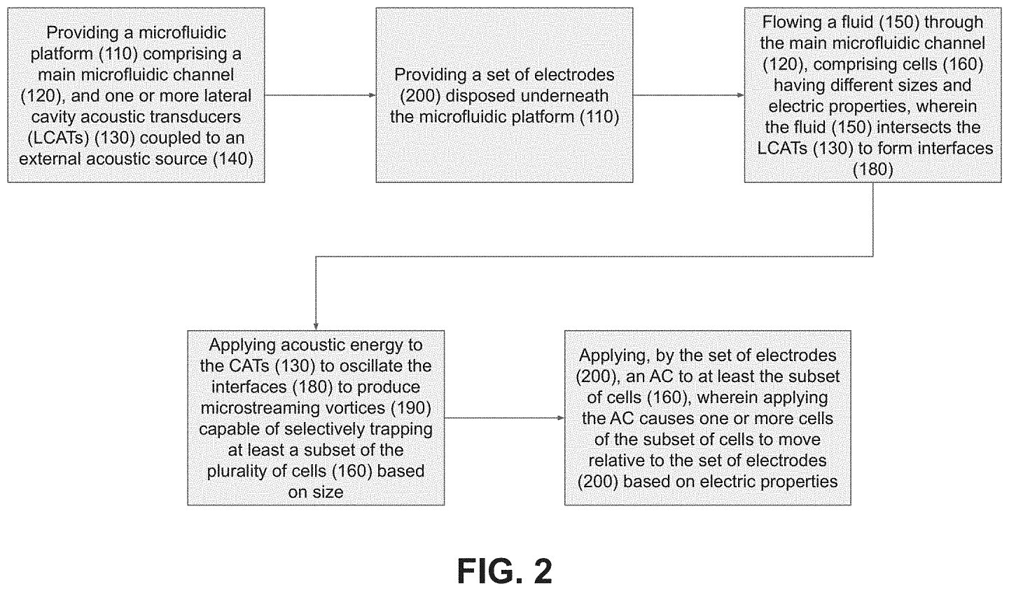

Near the air-liquid interfaces of the CAT, there may be a narrow gap where streamlines are directed forward in the direction of the bulk of the flow (see FIG. 2B). Particles whose diameter is bigger than this gap will tend to get pulled into the vortices, thus getting trapped. Different operation conditions (Piezo voltage, for example) affect the shape and stability of the vortices. In some embodiments, a shape of the microchannel may also affect the narrow gap, thus affecting particle sorting.

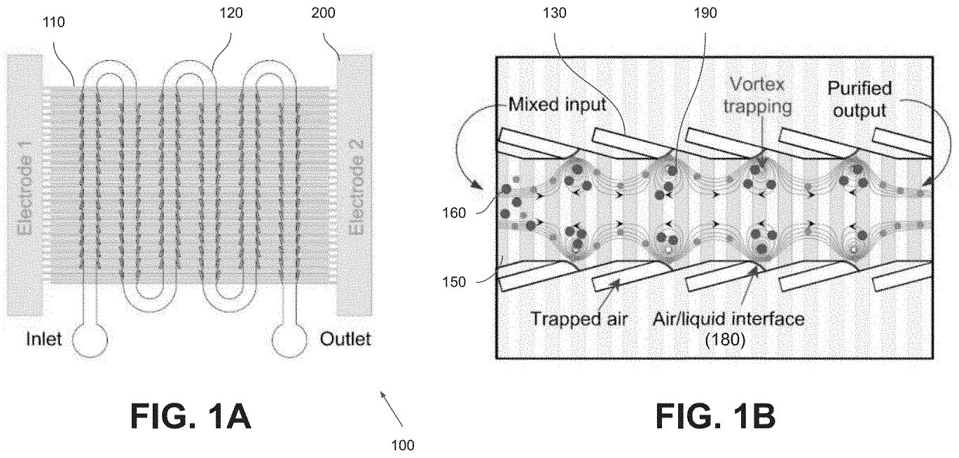

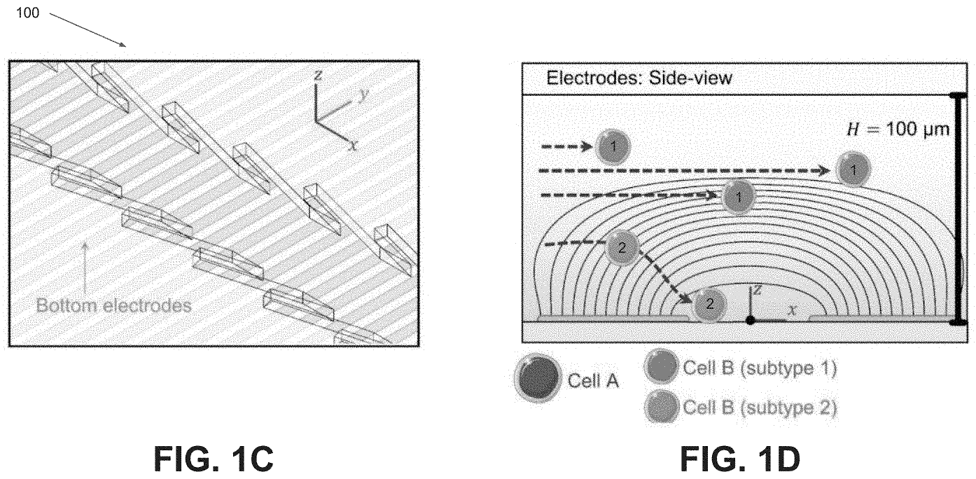

Referring now to FIGS. 1A-1D, the present invention features a system (100) for high-throughput cell sorting. The system (100) may comprise a microfluidic platform (110) may comprise a main microfluidic channel (120), and a microvortex generation component fluidly coupled to the microfluidic platform (110). In some embodiments, the microvortex generation component may comprise one or more CATs (130). The one or more CATs (130) may be dead-end channels coupled to the main microfluidic channel (120). The microfluidic platform (110) may be coupled to an external acoustic source (140). In some embodiments, the microvortex generation component may further comprise an external pump. In this embodiment, the external pump may pump the bulk of the fluid (150) through the main microfluidic channel (120) while the CATs (130) may produce one or more microstreaming vortices (190). The system (100) may further comprise a fluid (150) disposed through the main microfluidic channel (120). Said fluid (150) may comprise a plurality of cells (160) having different sizes and different electric properties. The fluid (150) may intersect the CATs (130) to form one or more interfaces (180). The system may further comprise a set of electrodes (200) disposed on a floor of the main microfluidic channel (120). In some embodiments, the system (100) may further comprise a second set of electrodes disposed on a wall or a ceiling of the main microfluidic channel (120). The set of electrodes (200) may be configured to apply an alternating current (AC) to the plurality of cells (160). The CATs (130) may be configured to oscillate the interfaces (180) to produce one or more microstreaming vortices (190), such that each microstreaming vortex may be capable of selectively trapping at least a subset of the plurality of cells (160) based on size. Applying the AC to the plurality of cells (160) may cause the plurality of cells (160) to move relative to the set of electrodes (200) based on electric properties through dielectric polarization. In some embodiments, the CATs (130) may be disposed laterally to or on top of the main channel (120).

In some embodiments, the plurality of cells may comprise one or more cell subtypes. The general idea is that CAT vortices allow size-based separation, while application of the AC-signal (either intermittently or continuously) can move cell subtypes relative to the overall device flow depending on their electric properties. The net result is that a cell subtype can be extracted at the outlet of the device, even when the different cell subtypes could have an overlapping size distribution.

In some embodiments, the oscillation may be controlled by a piezoelectric transducer (PZT) voltage. In some embodiments, the CATs (130) may be configured to induce pumping of the fluid (150), thereby eliminating the need for external pumping. In some embodiments, the interfaces (180) may comprise a gas-liquid interface, a liquid-liquid interface, a lipid membrane, a polymer membrane, a nano-particle membrane, or a combination thereof. In some embodiments, the system (100) may be used to purify cell mixtures that may comprise different subpopulations with overlapping sizes. For example, the system (100) may be used to separate lymphocyte subtypes in a high throughput manner for biomedical applications. In some embodiments, the electrodes (200) may be disposed parallel or perpendicular to a flow direction of the main microfluidic channel (120).

Referring now to FIG. 2, the present invention features a high-throughput method for cell sorting. The method may comprise providing a microfluidic platform (110). The microfluidic platform (110) may comprise a main microfluidic channel (120) and a microvortex generation component fluidly coupled to the microfluidic platform (110). In some embodiments, the microvortex generation component may comprise one or more CATs (130). The one or more CATs (130) may be dead-end channels coupled to the main microfluidic channel (120). The microfluidic platform (110) may be coupled to an external acoustic source (140). In some embodiments, the microvortex generation component may further comprise an external pump. In this embodiment, the external pump may pump the bulk of the fluid (150) through the main microfluidic channel (120) while the CATs (130) may produce one or more microstreaming vortices (190). The method may further comprise providing a set of electrodes (200) disposed on a floor of the main microfluidic channel (120). In some embodiments, the system (100) may further comprise a second set of electrodes disposed on a wall or a ceiling of the main microfluidic channel (120). The method may further comprise flowing a fluid (150) through the main microfluidic channel (120). Said fluid (150) may comprise a plurality of cells (160) having different sizes and different electric properties. The fluid (150) may intersect the CATs (130) to form one or more interfaces (180). The method may further comprise applying acoustic energy to the CATs (130) via the external acoustic source (140) to oscillate the interfaces (180). Oscillating the interfaces (180) may produce one or more microstreaming vortices (190) such that each microstreaming vortex may be capable of selectively trapping at least a subset of the plurality of cells (160) based on size. The method may further comprise applying, by the set of electrodes (200), an AC to the plurality of cells (160). Applying the AC may cause the plurality of cells (160) to move relative to the set of electrodes (200) based on electric properties through dielectric polarization. In some embodiments, the CATs (130) may be disposed laterally to or on top of the main channel (120).

In some embodiments, the oscillation may be controlled by a piezoelectric transducer (PZT) voltage. In some embodiments, the CATs (130) may be configured to induce pumping of the fluid (150), thereby eliminating the need for external pumping. In some embodiments, the interfaces (180) may comprise a gas-liquid interface, a liquid-liquid interface, a lipid membrane, a polymer membrane, a nano-particle membrane, or a combination thereof. In some embodiments, the method may be used to purify cell mixtures that may comprise different subpopulations with overlapping sizes. For example, the method may be used to separate lymphocyte subtypes in a high throughput manner for biomedical applications. In some embodiments, the electrodes (200) may be disposed parallel or perpendicular to a flow direction of the main microfluidic channel (120).

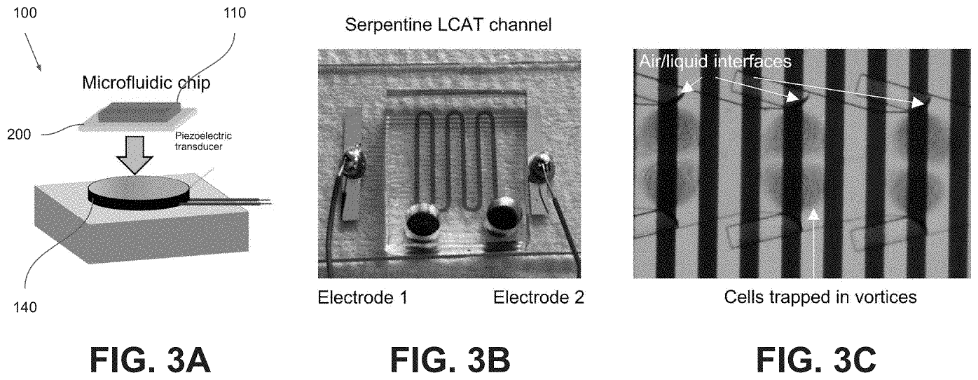

The present invention features a microfluidic platform that incorporates two techniques to improve cell separation sensitivity without compromising particle sorting throughput. The device may implement Cavity Acoustic Transducers (CATs) and Dielectrophoresis (DEP). The device may comprise a microfluidic channel that has lateral air-liquid interfaces that may be actuated by a piezoelectric transducer placed below the chip (see FIGS. 3A-3B). The actuation results in the oscillation of the air/liquid interface, which may produce flow vortices that may selectively trap cells by size (see FIG. 3C). A set of electrodes may be placed at the bottom of the microfluidic device (see FIGS. 1A & 1C), which may then be used to apply an AC electric field. As cells become dielectrically polarized in this field, they may experience a net movement (i.e., dielectrophoresis) relative to the electrodes (see FIG. 1D). This force may be highly dependent on the intrinsic electrical properties of cells, and therefore may be used to attract cells of a specific electric phenotype.

Using the above-described technologies, the separation works by first switching on the LCAT, which induces the vortex formation and cell trapping by size. After trapping cells of a specific size range in the vortices by this method, the LCAT is switched off and the DEP electrodes on, so that only cells with specific electric properties are attracted towards the electrodes—a mode known as positive DEP (FIG. 1D). Finally, cells that were not pulled towards the electrodes are flushed and collected at the device outlet.

The remaining cells can also be subsequently collected by switching the field off. The present invention features interplay between acoustic streaming sorting and DEP separation. In some embodiments, the present invention features turning off the acoustic transducer so that the cells settle on the electrodes and are exposed to greater DEP forces. Otherwise, cells that are trapped and swirling in the microfluidic vortices cannot be pulled with sufficient DEP force to the electrodes. A default operation would be to pump cells in and enrich for certain cell size subpopulation via the LCAT, turning off the LCAT so cells settle and DEP is turned on to trap cells with certain dielectric properties, while keeping the DEP force on then turning on the LCAT (or external pump) to remove cells not attracted by DEP electrodes. Likely there would be an on and off switching algorithm that optimizes the interplay between DEP force and acoustic streaming shear force.

The present invention may allow for the separation of lymphocyte subtypes in a high throughput manner. Lymphocytes represent an important component of the adaptive immune system and can be classified into two major classes: B lymphocytes (B-cells) and T lymphocytes (T-cells). T-cells are of particular interest, as they help control the body's immune response to foreign substances, and they destroy cells that have been infected by viruses or become cancerous. Isolating T-cells is critical to studying the body's immune response and is directly applicable to the development of medicines for the treatment of infectious diseases and cancer in the pharmaceutical industry. In some embodiments, the plurality of cells comprise peripheral blood mononuclear cells (which includes monocytes and lymphocytes). Thus the technology could be configured for monocytes with size based separation (CATs), while allowing for refined sorting of B vs. T cells (which have overlapping sizes).

Lymphocyte subpopulations possess different intrinsic electrokinetic properties that have been leveraged to separate and detect them in a label-free manner. For instance, T and B-cells have different electrophoretic mobilities and membrane capacitance. Despite the existence of label-free lymphocyte subtype characterization techniques, there is still a great need for gentle, efficient, and scalable lymphocyte isolation methods from whole blood for further downstream analysis, and processing. The proposed invention implements LCAT devices, which have been proven to be an effective tool for gentle, size-based separation of leukocytes from whole blood. This technology is then combined with the electrophysiological specificity of DEP methods, which have been proven to be effective in the separation of cells with identical sizes, yet different electric signatures.

When compared with other sorting methods such as inertial microfluidics and conventional acoustofluidic devices that rely mostly on size differences, the present invention provides more sensitive and specific isolation of the cell subtypes of interest. Thus, the combination of LCAT and DEP improves the separation capabilities of each of these techniques alone. This is because DEP provides a more sensitive separation that does not rely on size alone to achieve the separation (i.e., it relies on the intrinsic electrophysiological phenotype of cells). Furthermore, the addition of LCAT to a DEP-based method increases the throughput that DEP methods can usually reach alone. These benefits may have the added advantages provided by LCAT: 1) A label-free trapping of cells; 2) Optimized cell viability; 3) Ease of operation, as no external pumping systems are needed; and 4) competitive throughput for the handling of relevant whole blood cell concentrations.

In some embodiments, the CATs (130) may intersect the main channel (120) at an angle. As a non-limiting example, the angle may be between about 40-50 degrees. In other embodiments, the angle may be about 1-10, 10-20, 20-30, 30-40, 50-60, 60-70, 70-80, or 80-90 degrees.

In some embodiments, the electrodes (200) may be used to selectively collect, concentrate, and detect intracellular components from purified cells trapped in microstreaming vortices (190). In some embodiments, the plurality of cells (160) may be lysed by the electrodes (200), pumping a lysing buffer into the system, or a combination thereof. In some embodiments, the intracellular component may comprise DNA, RNA, protein, a small molecule, or a combination thereof. In some embodiments, the electrodes (200) may be used for Polymerase Chain Reaction (PCR) heating of concentrated nucleic acids.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A system (100) for high-throughput cell sorting, comprising:
a. a microfluidic platform (110) comprising a main microfluidic channel (120);
b. a microvortex generation component fluidly coupled to the microfluidic platform (110);
c. a fluid (150) disposed through the main microfluidic channel (120), said fluid (150) comprising a plurality of cells (160) having different sizes and different electric properties; and
d. a set of electrodes (200) disposed on a floor of the main microfluidic channel (120), wherein the set of electrodes (200) is configured to apply an alternating current (AC) to the plurality of cells (160);
wherein the microvortex generation component is configured to produce one or more microstreaming vortices, wherein each microstreaming vortex is capable of selectively trapping at least a subset of the plurality of cells (160) based on size;
wherein applying the AC to the plurality of cells (160) causes the plurality of cells (160) to move relative to the set of electrodes (200) based on the electric properties through dielectric polarization.

2. The system of claim 1, wherein the oscillation is controlled by a piezoelectric transducer (PZT) voltage.

3. The system of claim 1, wherein the interfaces (180) comprise a gas-liquid interface, a liquid-liquid interface, a lipid membrane, a polymer membrane, a nano-particle membrane, or a combination thereof.

4. The system of claim 1, wherein the system (100) is used to purify cell mixtures comprising different subpopulations with overlapping sizes.

5. The system of claim 4, wherein the system (100) is used to separate lymphocyte subtypes in a high throughput manner for biomedical applications.

6. The system of claim 1, wherein the electrodes (200) are disposed parallel or perpendicular to a flow direction of the main microfluidic channel (120).

7. The system of claim 1, wherein the microvortex generation component comprises one or more cavity acoustic transducers (CATs) (130), wherein the one or more CATs (130) are dead-end channels coupled to the main microfluidic channel (120), wherein the microfluidic platform (110) is coupled to an external acoustic source (140), wherein the fluid (150) intersects the CATs (130) to form one or more interfaces (180), wherein the CATs (130) are configured to oscillate the interfaces (180) to produce one or more microstreaming vortices (190).

8. The system of claim 7, wherein the CATs (130) are disposed laterally to or on top of the main channel (120).

9. The system of claim 1, wherein the microvortex generation component comprises an external pump.

10. A high-throughput method for cell sorting, comprising:
a. providing a microfluidic platform (110) comprising a main microfluidic channel (120);
b. providing a microvortex generation component fluidly connected to the microfluidic platform (110);
c. providing a set of electrodes (200) disposed on a floor of the main microfluidic channel (120);
d. flowing a fluid (150) through the main microfluidic channel (120), said fluid (150) comprising a plurality of cells (160) having different sizes and different electric properties:
e. producing, by the microvortex generation component, one or more microstreaming vortices (190) such that each microstreaming vortex is capable of selectively trapping at least a subset of the plurality of cells (160) based on size:
f. applying, by the set of electrodes (200), an alternating current (AC) to the plurality of cells (160), wherein applying the AC causes the plurality of cells (160) to move relative to the set of electrodes (200) based on the electric properties through dielectric polarization.

11. The method of claim 10, wherein the oscillation is controlled by a piezoelectric transducer (PZT) voltage.

12. The method of claim 10, wherein the interfaces (180) comprise a gas-liquid interface, a liquid-liquid interface, a lipid membrane, a polymer membrane, a nano-particle membrane, or a combination thereof.

13. The method of claim 10, wherein the method is used to purify cell mixtures comprising different subpopulations with overlapping sizes.

14. The method of claim 13, wherein the method is used to separate lymphocyte subtypes in a high throughput manner for biomedical applications.

15. The method of claim 10, wherein the electrodes (200) are disposed parallel or perpendicular to a flow direction of the main microfluidic channel (120).

16. The method of claim 10, wherein the microvortex generation component comprises one or more cavity acoustic transducers (CATs) (130), wherein the one or more CATs (130) are dead-end channels coupled to the main microfluidic channel (120), wherein the microfluidic platform (110) is coupled to an external acoustic source (140), wherein the fluid (150) intersects the CATs (130) to form one or more interfaces (180), wherein the CATs (130) are configured to oscillate the interfaces (180) to produce one or more microstreaming vortices (190).

17. The method of claim 16, wherein the CATs (130) are disposed laterally to or on top of the main channel (120).

18. The method of claim 10, wherein the microvortex generation component comprises an external pump.

19. The system of claim 1, wherein the electrodes (200) are used to selectively collect, concentrate, and detect intracellular components from purified cells trapped in microstreaming vortices (190).

20. The system of claim 1, wherein the plurality of cells (160) is lysed by the electrodes (200), pumping a lysing buffer into the system, or a combination thereof.

21. The system of claim 1, wherein the intracellular component comprises DNA, RNA, protein, a small molecule, or a combination thereof.

22. The system of claim 1, wherein the electrodes (200) are used for Polymerase Chain Reaction (PCR) heating of concentrated nucleic acids.
